(19) United States
(12) Patent Application Publication
(10) Pub. No.: US 2026/0179776 A1
(43) Pub. Date: Jun. 25, 2026

(54) HARDWARE-SECURED FEDERATED AI PLATFORM FOR MULTIMODAL NEURODEGENERATIVE RISK PREDICTION AND ADAPTIVE GOVERNANCE

(71) Applicant: George William Bickerstaff, III, Greenwich, CT (US)

(72) Inventor: George William Bickerstaff, III, Greenwich, CT (US)

(21) Appl. No.: 19/542,064

(22) Filed: Feb. 17, 2026

(51) Int. Cl. G16H 50/20; G06F 21/57; H04L 9/32

(52) U.S. Cl. CPC G16H 50/20; G06F 21/57; H04L 9/3278

(57) ABSTRACT

A platform provides cryptographically sovereign, hardware-rooted identity and execution authority for federated AI in neurodegenerative diagnostics. Each instance is assigned a permanent sovereign identity token bound to a hardware security module (HSM). AI model weights are stored in an encrypted state and only decrypted within a Trusted Execution Environment (TEE) upon token validation. A predicate-driven fusion lattice aligns multimodal data (genomic, imaging, behavioral) and applies biological constraints at the hardware level. Remote revocation instantly renders the platform inoperable by purging ephemeral decryption keys, ensuring immutable governance and clinical safety.

### FIELD OF THE INVENTION

The invention relates to a substantially resistant to bypass hardware-to-software integration for secure medical diagnostics. Specifically, it relates to a platform where Encrypted Model Weights are functionally inseparable from a Hardware Security Module (HSM), utilized for the predictive risk assessment of neurodegenerative disorders via a Lattice-Based Multimodal Fusion engine that enforces clinical safety constraints at the silicon level. This ensures that diagnostic AI operations are anchored in physical hardware, preventing unauthorized execution and enhancing patient safety in clinical environments.

### BACKGROUND OF THE INVENTION

Conventional medical AI frameworks suffer from “Software-Only Vulnerability,” where sensitive diagnostic models can be extracted, tampered with, or run on unauthorized hardware. Furthermore, existing federated learning models for neurodegenerative diseases (e.g., Alzheimer's, Parkinson's) operate as “Black Boxes,” lacking a verifiable mechanism to ensure that predictions remain within the bounds of biological reality (predicates). There remains a critical need for a system that anchors AI execution in a physical Root of Trust to ensure auditability, data sovereignty, and patient safety. Prior systems also fail to integrate hardware-enforced predicates that gate outputs based on clinical constraints, leading to potential errors in high-stakes diagnostics. Additionally, traditional approaches do not provide resilient state recovery in distributed networks, making them susceptible to downtime or inconsistent governance.

### SUMMARY OF THE INVENTION

The present invention provides a system where AI inference is cryptographically gated. By storing model parameters in an encrypted state that can only be decrypted within a Trusted Execution Environment (TEE) upon presentation of a Sovereign Identity Token, the system ensures that medical models cannot be leaked. The platform utilizes a Predicate-Driven Fusion Lattice to align multimodal inputs (genomic, imaging, behavioral) while applying hard-coded clinical constraints to neural network outputs, ensuring diagnostic defensibility and adaptive governance. This framework includes mechanisms for swarm-based propagation and state recovery, allowing nodes to re-validate through peer quorum without central authority. Overall, the invention delivers a secure, resilient platform for neurodegenerative risk prediction that prioritizes clinical accuracy and regulatory compliance. Remote revocation is achieved through a ledger-based disable mechanism, where a signed Revocation Signal propagates across the network to purge ephemeral decryption keys and mark the affected node as invalid in the Remote Revocation Ledger. In certain embodiments, the platform further computes Stakeholder Alignment Indices derived from the multimodal fusion lattice to quantify cross-modal evidentiary coherence and support transparent resource allocation, auditability, and collaborative clinical decision-making.

### Definitions

Encrypted Weight Orchestration—The process by which AI model parameters remain ciphertext until a hardware-rooted key enables ephemeral decryption within protected memory.

Hardware-Anchored Execution—Device-bound gating of AI functions using validated tokens, implemented in secure enclaves to block unauthorized actions.

Multimodal Fusion Lattice—An attention-weighted structure aligning diverse data streams with predicate constraints for unified representations.

Predicate-Constrained Fusion—A mathematical alignment where a logic-based “Safe-to-Act” gate nullifies neural outputs that violate biological bounds.

Sovereign Identity Token—A cryptographically signed identifier permanently bound to a physical device's silicon at manufacture or first boot.

Stakeholder Alignment Indices—Quantitative metrics derived from the fusion lattice that evaluate the compatibility of multimodal data streams with predefined neurodegenerative risk profiles, facilitating optimized resource allocation in collaborative settings.

### Distinction from Prior Art

Federated learning systems for medical diagnostics provide privacy-preserving training but do not bind execution to hardware-rooted tokens or enforce predicate-constrained fusion for neuro-specific risks. Blockchain-ledger approaches offer traceability but lack substantially resistant to bypass gates and hybrid classifiers tailored to multimodal neuro data. Conventional adaptive AI lacks quorum-governed adaptations with resilience metrics. The present invention applies hardware security in a novel manner specifically designed for predictive neurodegenerative governance, addressing vulnerabilities in prior systems by integrating encrypted weights and predicate gates.

### DETAILED IMPLEMENTATION EMBODIMENTS

### Example 1—Clinical Deployment for Alzheimer's Prediction

In a hospital setting, the platform processes diverse cases from enhanced datasets, yielding reliable early detection outcomes. Hardware anchors safeguard model integrity in federated updates across sites, while predicate classifiers identify gait irregularities via wearables in real time. Drift events prompt ledger consensus, preserving compliance. Clinicians utilize Predictive NeuroRisk Scores for targeted interventions, elevating patient care. Seamless integration with health records streamlines operations. Continuous refinements adapt to emerging patterns securely.

### Example 2—Research Coalition Optimization

In multi-institution Parkinson's studies, the system merges EEG and genomic inputs, generating Stakeholder Alignment Indices for optimal team assembly. Resilience metrics model breach scenarios, with swarm protocols neutralizing threats promptly. Outputs with provenance direct trial designs. Researchers exploit the fusion lattice for novel pattern discovery, advancing understanding of neurodegenerative processes. Ledger trails offer transparent documentation for funding and dissemination. Collaboration tools facilitate instant insight exchange, supporting research progress.

Biological predicates are encoded using a combination of rule engines, lookup tables, and hardware-implemented logic gates within the HSM. Rule engines evaluate conditional statements against clinical thresholds (e.g., biomarker ranges), lookup tables store predefined biological bounds for rapid validation, and logic gates perform bitwise operations to enforce nullification of invalid outputs, ensuring that all predicate checks are executed at the silicon level for efficiency and security.

### BRIEF DESCRIPTION OF THE DRAWINGS

FIG. 1 FRAMEWORK OVERVIEW

FIG. 2 INTEGRATION PIPELINE

FIG. 3 ADAPTATION LOOP

FIG. 4 VISUALIZATION INTERFACE

FIG. 5 BENCHMARKING MODULE

### DETAILED DESCRIPTION OF THE DRAWINGS

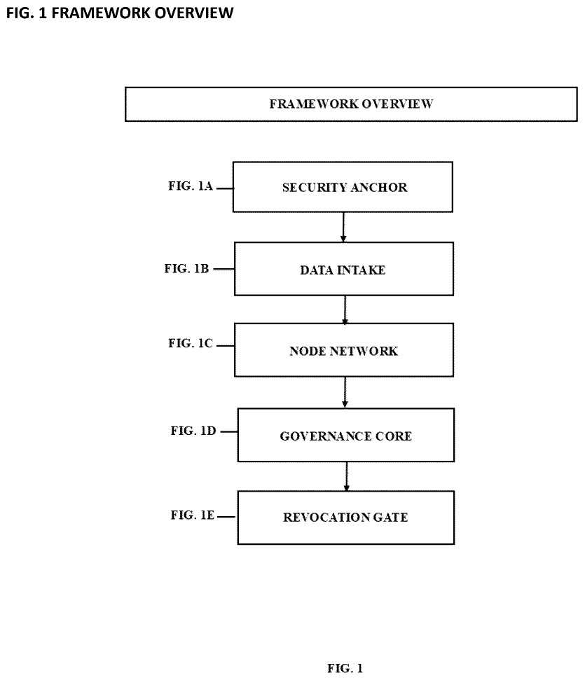

### FIG. 1 Framework Overview

FIG. 1 illustrates the complete system architecture, depicting the interconnected modules that form the backbone of the hardware-secured federated AI platform, including the integration of security anchors, data intake, node networks, governance cores, and revocation gates. This figure provides a high-level blueprint of how hardware anchors interact with software modules to maintain cryptographic integrity across the distributed network, emphasizing features such as scalability through node expansion and redundancy via quorum-based validation to handle failures in clinical environments. It serves as the foundational diagram for understanding the system's end-to-end architecture, where each subfigure delves into specific elements that contribute to the framework's sovereignty, reliability, and ability to enforce patient data protection while enabling seamless federated learning for neurodegenerative risk assessment.

### FIG. 1A Security Anchor

FIG. 1A depicts the Hardware Security Anchor, which generates sovereign identity tokens bound irreversibly to device modules using physical unclonable functions (PUFs) derived from silicon variations, enforcing execution gating to prevent unauthorized operations even in compromised environments. This anchor employs elliptic curve cryptography for token creation, ensuring that all actions authenticate against the root of trust by verifying digital signatures and preventing replay attacks through nonce inclusion. Integration with physical enclaves provides tamper resistance, defending against sophisticated attacks in medical settings by incorporating anti-side-channel protections and secure boot mechanisms that validate firmware integrity at startup.

### FIG. 1B Data Intake

FIG. 1B shows the Data Sovereignty Intake module, encrypting multimodal streams from clinical sources such as MRI scans, genomic sequences, and wearable sensor data while validating integrity and privacy through hash-based message authentication codes (HMACs). End-to-end protocols protect transmissions, compliant with standards like HIPAA by utilizing AES-256 encryption and differential privacy techniques to anonymize sensitive patient information during ingress. Consensus corroboration filters anomalies, enhancing trustworthiness before core processing by employing Byzantine fault-tolerant algorithms to cross-verify data from multiple nodes and discard outliers that could indicate tampering or errors.

### FIG. 1C Node Network

FIG. 1C illustrates the Federated Node Network, enabling decentralized training without raw data sharing by aggregating model gradients securely across geographically distributed hospital and research sites, fostering confidential collaboration. Nodes synchronize parameters securely, mitigating breach risks in distributed setups through homomorphic encryption that allows computations on ciphertext without decryption. Architecture allows node scalability, suitable for expansive neuro research consortia by supporting dynamic addition of nodes via peer discovery protocols and load balancing to maintain performance during high-volume data processing.

### FIG. 1D Governance Core

FIG. 1D presents the Ledger Governance Core, logging events in verifiable chains for regulatory audits using an append-only blockchain structure that records model updates, access attempts, and predicate validations with timestamps. Automatic recording with signatures ensures transparency and non-repudiation by requiring multi-party approvals for changes and employing zero-knowledge proofs to verify compliance without revealing underlying data. Quorum votes arbitrate changes, maintaining defensible oversight through threshold cryptography where a majority of trusted nodes must consensus on adaptations to prevent unauthorized modifications.

### FIG. 1E Revocation Gate

FIG. 1E details the Revocation Gate, invalidating compromised elements network-wide upon threat detection by monitoring anomaly scores from drift detectors and triggering global signals. Swarm protocols disseminate signals efficiently, minimizing critical downtime by using gossip-based propagation to ensure rapid delivery even in partially connected networks. Hardware blocks provide fail-safe protection against risks by physically disabling decryption capabilities in the HSM until re-validation, incorporating self-destruct mechanisms for keys in extreme breach scenarios.

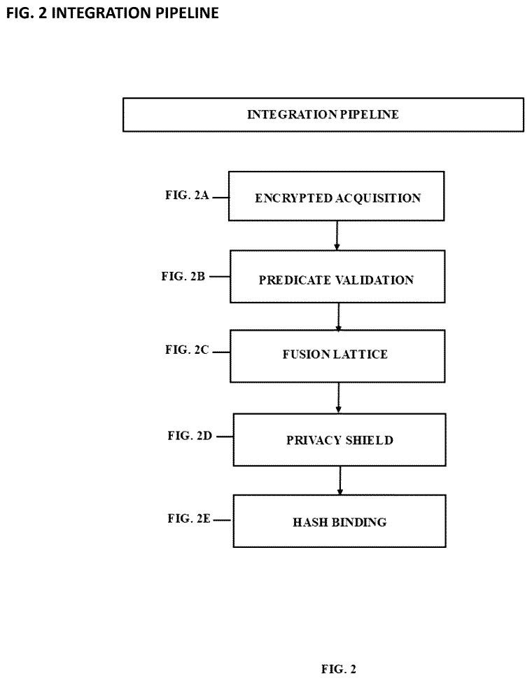

### FIG. 2 Integration Pipeline

FIG. 2 outlines the multimodal data integration pipeline, demonstrating sequential flow from acquisition to fusion, focused on secure processing of inputs like brain imaging, genetic markers, and behavioral logs. This figure emphasizes harmonizing modalities for cohesive spaces crucial to neuro predictions by applying attention mechanisms to weigh data relevance based on clinical correlations. It encapsulates data handling, with subfigures revealing privacy and extraction mechanisms that ensure end-to-end security through encrypted channels and predicate checks at each stage.

### FIG. 2A Encrypted Acquisition

FIG. 2A portrays the Encrypted Acquisition stage, collecting data like scans and metrics through secure channels that employ transport layer security (TLS) with forward secrecy to protect against eavesdropping. Algorithms safeguard transit, ensuring regulatory compliance by integrating access controls that limit data exposure to authorized nodes only. Quality checks verify integrity before advancement using checksums and machine learning-based anomaly detection to flag corrupted or malicious inputs early in the pipeline.

### FIG. 2B Predicate Validation

FIG. 2B describes the Predicate Validation component, applying rules for anomaly flagging based on predefined biological thresholds such as amyloid beta levels or neural activation patterns. Adaptive thresholds enhance accuracy, reducing clinical false positives by dynamically adjusting based on historical data distributions and feedback loops from prior predictions. Hardware integration ensures corroborated data proceeds by offloading validation to the HSM, where logic gates enforce real-time checks without exposing sensitive computations to the host OS.

### FIG. 2C Fusion Lattice

FIG. 2C illustrates the Fusion Lattice, merging modalities via weighting and alignments that use tensor decompositions to create unified embeddings from disparate data types. Correlations enable progression insights by identifying patterns like genetic-imaging links in Alzheimer's pathology through cross-modal attention layers. Dynamic adjustments handle resolutions, versatile for datasets by scaling lattice dimensions automatically to accommodate varying input sizes and qualities.

### FIG. 2D Privacy Shield

FIG. 2D shows the Privacy Shield module, adding noise to gradients during fusion via techniques like Gaussian differential privacy to obscure individual contributions in federated aggregates. Compliance preserves utility for collaborations by calibrating noise levels to balance privacy with model accuracy, ensuring minimal performance degradation. Logging interventions supports audits by recording privacy parameters and their impacts in the provenance ledger for traceability.

### FIG. 2E Hash Binding

FIG. 2E depicts the Hash Binding process, sealing artifacts to chains for trails using SHA-256 hashes linked to blockchain entries for immutable provenance. Fingerprints facilitate source traceability by allowing quick verification of data origins and modifications through merkle tree structures. Sovereignty ensures detectability of tampering by embedding device-specific tokens in hashes, alerting the system to any discrepancies during audits.

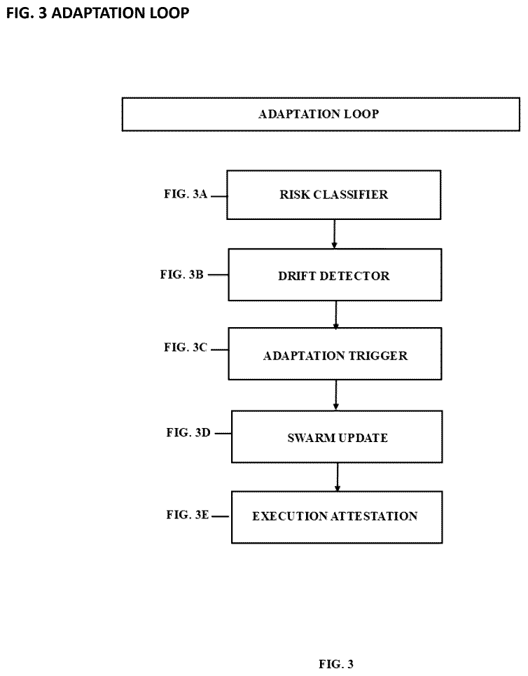

### FIG. 3 Adaptation Loop

FIG. 3 represents the predicate inference and adaptation loop, showcasing cyclical classification, monitoring, and refinement processes that iteratively improve model accuracy over time. This figure highlights adaptability with security, vital for neuro patterns by incorporating drift detection to respond to evolving disease biomarkers. It provides a comprehensive view of inference, with subfigures illustrating predicate and learning integration that ensures outputs remain clinically defensible.

### FIG. 3A Risk Classifier

FIG. 3A details the Risk Classifier, computing Scores via rules quantifying probabilities using hybrid neural-logic models that combine deep learning with symbolic reasoning. Hybrids achieve precision in detection by leveraging predicates to constrain probabilistic outputs within biological plausibility ranges. Calibrated outputs aid interventions by providing confidence intervals and explanations tied to input modalities for clinician review.

### FIG. 3B Drift Detector

FIG. 3B illustrates the Drift Detector, monitoring metrics for shifts in data distributions such as changes in sensor noise or biomarker norms over patient cohorts. Statistical measures trigger alerts proactively using Kolmogorov-Smirnov tests or Kullback-Leibler divergence to identify concept drift early. Ledger linkage upholds traceability by logging drift events with timestamps and affected nodes for forensic analysis.

### FIG. 3C Adaptation Trigger

FIG. 3C shows the Adaptation Trigger, aggregating improvements for approvals by collecting gradient updates from nodes and evaluating their impact on overall model stability. Voting validates, preventing modifications through multi-signature schemes requiring quorum agreement. Gated adaptation complies with guidelines by incorporating regulatory checks, such as HIPAA-aligned privacy reviews, before deployment.

### FIG. 3D Swarm Update

FIG. 3D presents the Swarm Update protocol, disseminating parameters securely via encrypted peer-to-peer channels that use onion routing for anonymity. Channels verify, minimizing latency by prioritizing updates based on node proximity and bandwidth. Rollback supports resilience by maintaining versioned model states in the ledger for quick reversion in case of faulty updates.

### FIG. 3E Execution Attestation

FIG. 3E depicts the Execution Attestation, verifying tokens before actions using remote attestation protocols like Intel SGX to prove enclave integrity. Proofs shareable with auditors by generating verifiable reports that include hashes of executed code and inputs. Gate blocks invalidated executions by revoking session keys immediately upon failed verification, ensuring no unauthorized inference occurs.

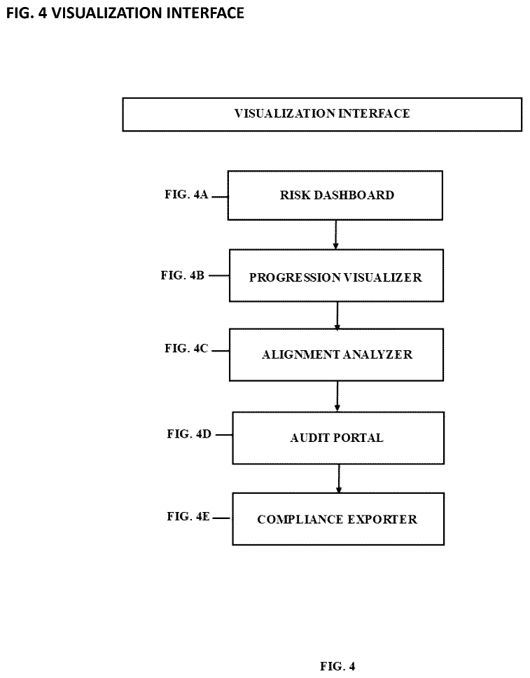

### FIG. 4 Visualization Interface

FIG. 4 captures the governance and visualization interface, illustrating user interactions with outputs and audits through interactive dashboards that display real-time risk scores and audit trails. This figure underscores translating insights into visuals with transparency by using heat maps and graphs to highlight key metrics. It overviews components, demonstrating usability and support for clinicians and researchers in interpreting complex AI outputs.

### FIG. 4A Risk Dashboard

FIG. 4A portrays the Risk Dashboard, displaying Scores with heat-maps that color-code risk levels based on multimodal fusion results for quick visual assessment. Aggregates updates with alerts by integrating real-time notifications for drift or high-risk predictions. Links recommendations, enhancing workflows by providing clickable pathways to detailed reports and intervention suggestions tailored to patient profiles.

### FIG. 4B Progression Visualizer

FIG. 4B illustrates the Progression Visualizer, generating trend graphs that plot NeuroRisk Scores over time using longitudinal data from repeated assessments. Projects states for planning by employing predictive modeling to forecast disease trajectories based on current trends. Exports integrate with records by supporting FHIR-compatible formats for seamless incorporation into electronic health systems.

### FIG. 4C Alignment Analyzer

FIG. 4C shows the Alignment Analyzer, computing Indices for fit by evaluating how well multimodal data aligns with known neurodegenerative patterns. Sub-scores provide rankings by breaking down contributions from each modality, such as imaging versus genomics. Simulations predict performance by running hypothetical scenarios to test alignment under varying data conditions.

### FIG. 4D Audit Portal

FIG. 4D details the Audit Portal, accessing chains for histories through searchable interfaces that query the provenance ledger for specific events. Tools trace events verifiably using cryptographic proofs to confirm authenticity without full chain download. Reports support compliance by automating generation of formatted documents for regulatory submissions.

### FIG. 4E Compliance Exporter

FIG. 4E depicts the Compliance Exporter, automating documents by compiling audit logs, privacy metrics, and validation proofs into standardized templates. Formats interoperable, streamlining submissions by supporting PDF, XML, and FHIR exports. Signatures ensure verifiability by embedding digital certificates from the HSM for non-repudiation.

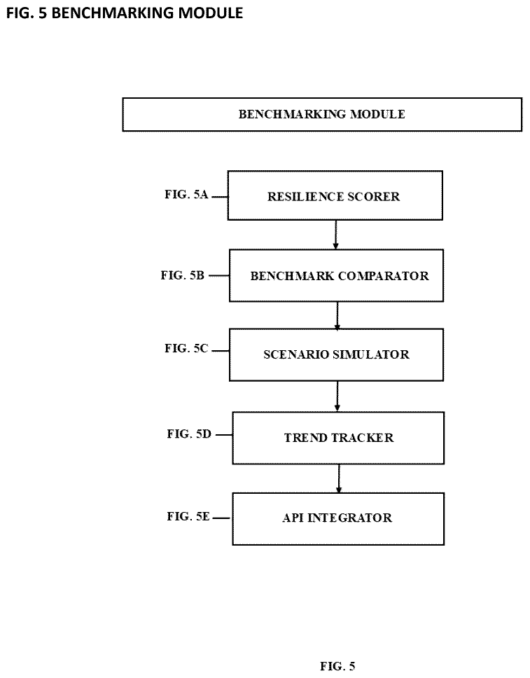

### FIG. 5 Benchmarking Module

FIG. 5 delineates the resilience and benchmarking module, evaluating stability and analyses through simulations of network failures and data perturbations. This figure emphasizes contribution to reliability in settings by providing metrics that guide optimizations in federated environments. It summarizes workflow, highlighting governance optimization by integrating resilience scores into adaptation decisions.

### FIG. 5A Resilience Scorer

FIG. 5A illustrates the Resilience Scorer, calculating Metrics against variations such as node dropouts or cyber threats using Monte Carlo simulations. Algorithms derive strategies by recommending redundancy levels or encryption strengths based on scored vulnerabilities. Ledger logging provides basis by archiving all scoring runs for historical trend analysis and compliance.

### FIG. 5B Benchmark Comparator

FIG. 5B shows the Benchmark Comparator, evaluating against cohorts by comparing performance metrics like accuracy and latency across similar federated systems. Mapping ranks fairly by normalizing scores to account for dataset differences and hardware variations. Customizations value comparisons by allowing user-defined weights for metrics like privacy versus speed.

### FIG. 5C Scenario Simulator

FIG. 5C depicts the Scenario Simulator, testing under conditions like data scarcity or high noise using synthetic data generation. Predicts with augmentation by applying adversarial examples to assess robustness against attacks. Reports facilitate decisions by visualizing outcomes in graphs and tables for easy interpretation.

### FIG. 5D Trend Tracker

FIG. 5D presents the Trend Tracker, monitoring evolutions in model performance over iterations using time-series analysis. Analysis forecasts issues by employing ARIMA models to predict potential drifts or degradations. Archives ensure insights by storing tracked data in compressed, searchable formats within the ledger.

### FIG. 5E API Integrator

FIG. 5E details the API Integrator, providing access conformant to standards like RESTful endpoints with OAuth authentication. Authentication prevents intrusions by enforcing token-based access controls tied to sovereign identities. Deployments enhance versatility by supporting containerized setups for cloud or on-premise integration.

1. A hardware-secured system for neurodegenerative risk prediction, comprising a Hardware Security Module (HSM) containing a non-volatile memory storing a unique Sovereign Identity Token bound to the device silicon; a Model Repository storing AI model weights in an encrypted ciphertext state; a Trusted Execution Environment (TEE) communicatively coupled to the HSM, configured to receive the Sovereign Identity Token and verify its validity against a Remote Revocation Ledger, generate a session-specific decryption key derived from said Token, and decrypt the AI model weights only within the protected memory of the TEE; a Predicate-Logic Gate stored within the HSM that intercepts the output of the decrypted AI model and validates it against a set of biological boundary constraints before permitting transmission to a user interface.

2. A method for secure, decentralized neurodegenerative assessment, comprising initializing a local diagnostic node by binding a Genesis Block of model lineage and training data hashes to a hardware-rooted identity; executing a Multimodal Fusion Lattice that aligns behavioral sensor data with biochemical biomarkers using an attention-weighted tensor; applying a Hardware-Verified Predicate Mask to the aligned data to filter out neural outputs that deviate from clinically defined neurodegenerative patterns; broadcasting a cryptographically signed Stability Metric to a federated network via a Swarm Propagation Protocol; and automatically rendering the local node inoperable by purging session-specific decryption keys upon receipt of a signed Revocation Signal.

3. A non-transitory computer-readable medium storing instructions that, when executed by a hardware security module, cause the system to perform the method of claim 2.

4. The system of claim 1, wherein the Sovereign Identity Token is irreversibly bound to the device's hardware via a physical unclonable function (PUF).

5. The system of claim 1, wherein the Predicate-Logic Gate nullifies risk scores if the input modality variance exceeds a hardware-stored threshold.

6. The method of claim 2, further comprising the step of recording every model adaptation event on an append-only, tamper-resistant Provenance Ledger.

7. The system of claim 1, wherein the execution control mechanism is substantially resistant to bypass even if the host operating system is compromised.

8. The system of claim 1, wherein the Predicate-Logic Gate triggers a “Conflict Audit” on the Ledger if predicted risk exceeds a predefined threshold but supporting evidence falls within normal ranges.

9. The method of claim 2, further comprising re-validation of a suspended node through quorum consensus from peer nodes in the federated network.

10. The system of claim 1, wherein the fusion lattice applies a hardware-verified attention mask to the multimodal data, such that data streams failing a hardware-stored predicate check are mathematically nullified within the risk prediction calculation, ensuring the Predictive NeuroRisk Score is derived only from corroborated evidence.

11. The method of claim 2, wherein the Swarm Propagation Protocol includes state recovery for offline nodes via encrypted broadcast and peer quorum verification.

12. The system of claim 1, wherein differential privacy is applied to gradients during federated aggregation.

13. The method of claim 2, wherein baseline updates use quantile mapping for percentile normalization.

14. The system of claim 1, wherein APIs conform to FHIR standards for interoperability.

15. The method of claim 2, wherein outputs include Stakeholder Alignment Indices.

16. The system of claim 1, wherein resilience metrics evaluate swarm stability.

17. The method of claim 2, wherein predicate rules filter environmental noise.

18. The system of claim 1, wherein the visualization interface includes longitudinal trajectory graphs.

19. The system of claim 1, wherein the session-specific decryption key is erased upon power cycle.
